SEQUENCE LISTING (16 sequences)

SEQ ID NO: 1 - DNA, 3036 bp, Homo sapiens
  misc_feature (1)..(79): >gi|178676|gb|M17303.1|HUMANTCE Human carcinoembryonic antigen gene, complete cds
cgaccagcagaccagacagtcacagcagccttgacaaaacgttcctggaactcaagcacttctccacagaggaggacagagcagacagcagagaccatggagtctccctcggcccctccccacagatggtgcatcccctggcagaggctcctgctcacagcctcacttctaaccttctggaacccgcccaccactgccaagctcactattgaatccacgccgttcaatgtcgcagaggggaaggaggtgcttctacttgtccacaatctgccccagcatctttttggctacagctggtacaaaggtgaaagagtggatggcaaccgtcaaattataggatatgtaataggaactcaacaagctaccccagggcccgcatacagtggtcgagagataatataccccaatgcatccctgctgatccagaacatcatccagaatgacacaggattctacaccctacacgtcataaagtcagatcttgtgaatgaagaagcaactggccagttccgggtatacccggagctgcccaagccctccatctccagcaacaactccaaacccgtggaggacaaggatgctgtggccttcacctgtgaacctgagactcaggacgcaacctacctgtggtgggtaaacaatcagagcctcccggtcagtcccaggctgcagctgtccaatggcaacaggaccctcactctattcaatgtcacaagaaatgacacagcaagctacaaatgtgaaacccagaacccagtgagtgccaggcgcagtgattcagtcatcctgaatgtcctctatggcccggatgcccccaccatttcccctctaaacacatcttacagatcaggggaaaatctgaacctctcctgccatgcagcctctaacccacctgcacagtactcttggtttgtcaatgggactttccagcaatccacccaagagctctttatccccaacatcactgtgaataatagtggatcctatacgtgccaagcccataactcagacactggcctcaataggaccacagtcacgacgatcacagtctatgcagagccacccaaacccttcatcaccagcaacaactccaaccccgtggaggatgaggatgctgtagccttaacctgtgaacctgagattcagaacacaacctacctgtggtgggtaaataatcagagcctcccggtcagtcccaggctgcagctgtccaatgacaacaggaccctcactctactcagtgtcacaaggaatgatgtaggaccctatgagtgtggaatccagaacgaattaagtgttgaccacagcgacccagtcatcctgaatgtcctctatggcccagacgaccccaccatttccccctcatacacctattaccgtccaggggtgaacctcagcctctcctgccatgcagcctctaacccacctgcacagtattcttggctgattgatgggaacatccagcaacacacacaagagctctttatctccaacatcactgagaagaacagcggactctatacctgccaggccaataactcagccagtggccacagcaggactacagtcaagacaatcacagtctctgcggagctgcccaagccctccatctccagcaacaactccaaacccgtggaggacaaggatgctgtggccttcacctgtgaacctgaggctcagaacacaacctacctgtggtgggtaaatggtcagagcctcccagtcagtcccaggctgcagctgtccaatggcaacaggaccctcactctattcaatgtcacaagaaatgacgcaagagcctatgtatgtggaatccagaactcagtgagtgcaaaccgcagtgacccagtcaccctggatgtcctctatgggccggacacccccatcatttcccccccagactcgtcttacctttcgggagcgaacctcaacctctcctgccactcggcctctaacccatccccgcagtattcttggcgtatcaatgggataccgcagcaacacacacaagttctctttatcgccaaaatcacgccaaataataacgggacctatgcctgttttgtctctaacttggctactggccgcaataattccatagtcaagagcatcacagtctctgcatctggaacttctcctggtctctcagctggggccactgtcggcatcatgattggagtgctggttggggttgctctgatatagcagccctggtgtagtttcttcatttcaggaagactgacagttgttttgcttcttccttaaagcatttgcaacagctacagtctaaaattgcttctttaccaaggatatttacagaaaagactctgaccagagatcgagaccatcctagccaacatcgtgaaaccccatctctactaaaaatacaaaaatgagctgggcttggtggcgcgcacctgtagtcccagttactcgggaggctgaggcaggagaatcgcttgaacccgggaggtggagattgcagtgagcccagatcgcaccactgcactccagtctggcaacagagcaagactccatctcaaaaagaaaagaaaagaagactctgacctgtactcttgaatacaagtttctgataccactgcactgtctgagaatttccaaaactttaatgaactaactgacagcttcatgaaactgtccaccaagatcaagcagagaaaataattaatttcatgggactaaatgaactaatgaggataatattttcataattttttatttgaaattttgctgattctttaaatgtcttgtttcccagatttcaggaaactttttttcttttaagctatccacagcttacagcaatttgataaaatatacttttgtgaacaaaaattgagacatttacattttctccctatgtggtcgctccagacttgggaaactattcatgaatatttatattgtatggtaatatagttattgcacaagttcaataaaaatctgctctttgtatgacagaatacatttgaaaacattggttatattaccaagactttgactagaatgtcgtat

SEQ ID NO: 2 - DNA, 1513 bp, Homo sapiens
  misc_feature (1)..(65): >gi|40217850|ref|NM_002276.3| Homo sapiens keratin 19 (KRT19), mRNA
cgcccctgacaccattcctcccttcccccctccaccggccgcgggcataaaaggcgccaggtgagggcctcgccgctcctcccgcgaatcgcagcttctgagaccagggttgctccgtccgtgctccgcctcgccatgacttcctacagctatcgccagtcgtcggccacgtcgtccttcggaggcctgggcggcggctccgtgcgttttgggccgggggtcgcctttcgcgcgcccagcattcacgggggctccggcggccgcggcgtatccgtgtcctccgcccgctttgtgtcctcgtcctcctcgggggcctacggcggcggctacggcggcgtcctgaccgcgtccgacgggctgctggcgggcaacgagaagctaaccatgcagaacctcaacgaccgcctggcctcctacctggacaaggtgcgcgccctggaggcggccaacggcgagctagaggtgaagatccgcgactggtaccagaagcaggggcctgggccctcccgcgactacagccactactacacgaccatccaggacctgcgggacaagattcttggtgccaccattgagaactccaggattgtcctgcagatcgacaatgcccgtctggctgcagatgacttccgaaccaagtttgagacggaacaggctctgcgcatgagcgtggaggccgacatcaacggcctgcgcagggtgctggatgagctgaccctggccaggaccgacctggagatgcagatcgaaggcctgaaggaagagctggcctacctgaagaagaaccatgaggaggaaatcagtacgctgaggggccaagtgggaggccaggtcagtgtggaggtggattccgctccgggcaccgatctcgccaagatcctgagtgacatgcgaagccaatatgaggtcatggccgagcagaaccggaaggatgctgaagcctggttcaccagccggactgaagaattgaaccgggaggtcgctggccacacggagcagctccagatgagcaggtccgaggttactgacctgcggcgcacccttcagggtcttgagattgagctgcagtcacagctgagcatgaaagctgccttggaagacacactggcagaaacggaggcgcgctttggagcccagctggcgcatatccaggcgctgatcagcggtattgaagcccagctgggcgatgtgcgagctgatagtgagcggcagaatcaggagtaccagcggctcatggacatcaagtcgcggctggagcaggagattgccacctaccgcagcctgctcgagggacaggaagatcactacaacaatttgtctgcctccaaggtcctctgaggcagcaggctctggggcttctgctgtcctttggagggtgtcttctgggtagagggatgggaaggaagggacccttacccccggctcttctcctgacctgccaataaaaatttatggtccaagggaaaaaaaaaaaaaaaaaaaaaaaaaaaaaaaaaaaaaaaaaaaaaaaaaa

SEQ ID NO: 3 - DNA, 15720 bp, Homo sapiens
  misc_feature (1)..(83): >gi|4505284|ref|NM_002457.1| Homo sapiens mucin 2, intestinal/tracheal (MUC2), mRNA
caacccacaccgcccctgccagccaccatggggctgccactagcccgcctggcggctgtgtgcctggccctgtctttggcagggggctcggagctccagacagagggcagaacccgataccacggccgcaacgtctgcagcacctggggcaacttccactacaagaccttcgacggggacgtcttccgcttccccggcctctgcgactacaacttcgcctccgactgccgaggctcctacaaggaatttgctgtgcacctgaagcggggtccgggccaggctgaggcccccgccggggtggagtccatcctgctgaccatcaaggatgacaccatctacctcacccgccacctggctgtgcttaacggggccgtggtcagcaccccgcactacagccccgggctgctcattgagaagagcgatgcctacaccaaagtctactcccgcgccggcctcaccctcatgtggaaccgggaggatgcactcatgctggagctggacactaagttccggaaccacacctgtggcctctgcggggactacaacggcctgcagagctattcagaattcctctctgacggcgtgctcttcagtcccctggagtttgggaacatgcagaagatcaaccagcccgatgtggtgtgtgaggatcccgaggaggaggtggcccccgcatcctgctccgagcaccgcgccgagtgtgagaggctgctgaccgccgaggccttcgcggactgtcaggacctggtgccgctggagccgtatctgcgcgcctgccagcaggaccgctgccggtgcccgggcggtgacacctgcgtctgcagcaccgtggccgagttctcccgccagtgctcccacgccggcggccggcccgggaactggaggaccgccacgctctgccccaagacctgccccgggaacctggtgtacctggagagcggctcgccctgcatggacacctgctcacacctggaggtgagcagcctgtgcgaggagcaccgcatggacggctgtttctgcccagaaggcaccgtatatgacgacatcggggacagtggctgcgttcctgtgagccagtgccactgcaggctgcacggacacctgtacacaccgggccaggagatcaccaatgactgcgagcagtgtgtctgtaacgctggccgctgggtgtgcaaagacctgccctgccccggcacctgtgccctggaaggcggctcccacatcaccaccttcgatgggaagacgtacaccttccacggggactgctactatgtcctggccaagggtgaccacaacgattcctacgctctcctgggcgagctggccccctgtggctccacagacaagcagacctgcctgaagacggtggtgctgctggctgacaagaagaagaatgcggtggtcttcaagtccgatggcagtgtactgctcaaccagctgcaggtgaacctgccccacgtgaccgcgagcttctctgtcttccgcccgtcttcctaccacatcatggtgagcatggccattggcgtccggctgcaggtgcagctggccccagtcatgcaactctttgtgacactggaccaggcctcccaggggcaggtgcagggcctctgcgggaacttcaacggcctggaaggtgacgacttcaagacggccagcgggctggtggaggccacgggggccggctttgccaacacctggaaggcacagtcaacctgccatgacaagctggactggttggacgatccctgctccctgaacatcgagagcgccaactacgccgagcactggtgctccctcctgaagaagacagagaccccctttggcaggtgccactcggctgtggaccctgctgagtattacaagaggtgcaaatatgacacgtgtaactgtcagaacaatgaggactgcctgtgcgccgccctgtcctcctacgcgcgcgcctgcaccgccaagggcgtcatgctgtggggctggcgggagcatgtctgcaacaaggatgtgggctcctgccccaactcgcaggtcttcctgtacaacctgaccacctgccagcagacctgccgctccctctccgaggccgacagccactgtctcgagggctttgcgcctgtggacggctgcggctgccctgaccacaccttcctggacgagaagggccgctgcgtacccctggccaagtgctcctgttaccaccgcggtctctacctggaggcgggggatgtggtcgtcaggcaggaagaacgatgtgtgtgccgggatgggcggctgcactgtaggcagatccggctgatcggccagagctgcacggccccaaagatccacatggactgcagcaacctgactgcactggccacctcgaagccccgagccctcagctgccagacgctggccgccggctattaccacacagagtgtgtcagtggctgtgtgtgccccgacgggctgatggatgacggccggggtggctgcgtggtggagaaggaatgcccttgcgtccataacaacgacctgtattcttccggcgccaagatcaaggtggactgcaatacctgcacctgcaagagaggacgctgggtgtgcacccaggctgtgtgccatggcacctgctccatttacgggagtggccactacatcacctttgatgggaagtactacgactttgacggacactgctcctacgtggctgttcaggactactgcggccagaactcctcactgggctcattcagcatcatcaccgagaacgtcccctgtggcactacgggcgtcacctgctccaaggccatcaagatcttcatggggaggacggagctgaagttggaagacaagcaccgtgtggtgatccagcgtgatgagggtcaccacgtggcctacaccacgcgggaggtgggccagtacctggtggtggagtccagcacgggcatcatcgtcatctgggacaagaggaccaccgtgttcatcaagctggctccctcctacaagggcaccgtgtgtggcctgtgtgggaactttgaccaccgctccaacaacgacttcaccacgcgggaccacatggtggtgagcagcgagctggacttcgggaacagctggaaggaggcccccacctgcccagatgtgagcaccaaccccgagccctgcagcctgaacccgcaccgccgctcctgggccgagaagcagtgcagcatcctcaaaagcagcgtgttcagcatctgccacagcaaggtggaccccaagcccttctacgaggcctgtgtgcacgactcgtgctcctgtgacacgggtggggactgtgagtgcttctgctctgccgtggcctcctacgcccaggagtgtaccaaagagggggcctgcgtgttctggaggacgccggacctgtgccccatattctgcgactactacaaccctccgcatgagtgtgagtggcactatgagccatgtgggaaccggagcttcgagacctgcaggaccatcaacggcatccactccaacatctccgtgtcctacctggagggctgctacccccggtgccccaaggacaggcccatctatgaggaggatctgaagaagtgtgtcactgcagacaagtgtggctgctatgtcgaggacacccactacccacctggagcatcggttcccaccgaggagacctgcaagtcctgcgtgtgtaccaactcctcccaagtcgtctgcaggccggaggaaggaaagattcttaaccagacccaggatggcgccttctgctactgggagatctgtggccccaacgggacggtggagaagcacttcaacatctgttccattacgacacgcccgtccaccctgaccaccttcaccaccatcaccctccccaccacccccacctccttcaccactaccaccaccaccaccaccccgacctccagcacagttttatcaacaactccgaagctgtgctgcctctggtctgactggatcaatgaggaccaccccagcagtggcagcgacgacggtgaccgagaaccatttgatggggtctgcggggcccctgaggacatcgagtgcaggtcggtcaaggatccccacctcagcttggagcagcatggccagaaggtgcagtgtgatgtctctgttgggttcatttgcaagaatgaagaccagtttggaaatggaccatttggactgtgttacgactacaagatacgtgtcaattgttgctggcccatggataagtgtatcaccactcccagccctccaactaccactcccagccctccaccaaccacgacgaccacccttccaccaaccaccacccccagccctccaaccaccaccacaaccacccctccaccaaccaccacccccagccctccaataaccaccacgaccacccctctaccaaccaccactcccagccctccaataagcaccacaaccacccctccaccaaccaccactcccagccctccaaccaccactcccagccctccaaccaccactcccagccctccaacaaccaccacaaccacccctccaccaaccaccactcccagccctccaatgactacgcccatcactccaccagccagcactaccacccttccaccaaccaccactcccagccctccaacaaccaccacaaccacccctccaccaaccaccactcccagtcctccaacgactacgcccatcactccaccaaccagcactactacccttccaccaaccaccactcccagccctccaccaaccaccacaaccacccctccaccaaccaccactcccagccctccaacaaccaccactcccagtcctccaacaatcaccacaaccacccctccaccaaccaccactcccagccctccaacaacgaccacaaccacccctccaccaaccaccactcccagccctccaacgactacacccatcactccaccaaccagcactaccacccttccaccaaccaccactcccagccctccaccaaccaccacaaccacccctccaccaaccaccactcccagccctccaacaaccaccactcccagccctccaataaccaccacaaccacccctccaccaaccaccactcccagctctccaataaccaccactcccagccctccaacaaccaccatgaccaccccttcaccaaccaccacccccagctctccaataaccaccacaaccaccccttcctcaactaccactcccagccctccaccaaccaccatgaccaccccttcaccaaccaccactcccagccctccaacaaccaccatgaccacccttccaccaaccaccacttccagccctctaacaactactcctctacctccatcaataactcctcctacattttcaccattctcaacgacaacccctactaccccatgcgtgcctctctgcaattggactggctggctggattctggaaaacccaactttcacaaaccaggtggagacacagaattgattggagacgtctgtggaccaggctgggcagctaacatctcttgcagagccaccatgtatcctgatgttcccattggacagcttggacaaacagtggtgtgtgatgtctctgtggggctgatatgcaaaaatgaagaccaaaagccaggtggggtcatccctatggccttctgcctcaactacgagatcaacgttcagtgctgtgagtgtgtcacccaacccaccaccatgacaaccaccaccacagagaacccaactccgccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccgggccccccacccacacaagcacagcaccgattgctgagttgaccacatccaatcctccgcctgagtcctcaacccctcagacctctcggtccacctcttcccctctcacggagtcaaccacccttctgagtaccctaccacctgccattgagatgaccagcacggccccaccctccacacccacggcacccacgaccacgagcggaggccacacactgtctccaccgcccagcaccaccacgtcccctccaggcacccccactcgcggtaccacgaccgggtcatcttcagcccccacccccagcactgtgcagacgaccaccaccagtgcctggaccccaacgccgaccccactctccacacccagcatcatcaggaccacaggcctgaggccctacccttcctctgtgcttatctgctgtgtcctgaacgacacctactacgcaccaggtgaggaggtgtacaacggcacatacggagacacctgttatttcgtcaactgctcactgagctgtacgttggagttctataactggtcctgcccatccacgccctccccaacacccacgccctccaagtcgacgcccacgccttccaagccatcgtccacgccctccaagccgacgcccggcaccaagccccccgagtgcccagactttgatcctcccagacaggagaacgagacttggtggctgtgcgactgcttcatggccacgtgcaagtacaacaacacggtggagatcgtgaaggtggagtgtgagccgccgcccatgcccacctgctccaacggcctccaacccgtgcgcgtcgaggaccccgacggctgctgctggcactgggagtgcgactgctactgcacgggctggggcgacccgcactatgtcaccttcgacggactctactacagctaccagggcaactgcacctacgtgctggtggaggagatcagcccctccgtggacaacttcggagtttacatcgacaactaccactgcgatcccaacgacaaggtgtcctgtccccgcaccctcatcgtgcgccacgagacccaggaggtgctgatcaagaccgtgcatatgatgcccatgcaggtgcaggtgcaggtgaacaggcaggcggtggcactgccctacaagaagtacgggctggaggtgtaccagtctggcatcaactacgtggtggacatccccgagctgggtgtcctcgtctcctacaatggcctgtccttctccgtcaggctgccctaccaccggtttggcaacaacaccaagggccagtgtggcacctgcaccaacaccacctccgacgactgcattctgcccagcggggagatcgtctccaactgtgaggctgcggctgaccagtggctggtgaacgacccctccaagccacactgcccccacagcagctccacgaccaagcgcccggccgtcactgtgcccgggggcggtaaaacgaccccacacaaggactgcaccccatctcccctctgccagctcatcaaggacagcctgtttgcccagtgccacgcactggtgcccccgcagcactactacgatgcctgcgtgttcgacagctgcttcatgccgggctcgagcctggagtgcgccagtctgcaggcctacgcagccctctgtgcccagcagaacatctgcctcgactggcggaaccacacgcatggggcctgcttggtggagtgcccatctcacagggagtaccaggcctgtggccctgcagaagagcccacgtgcaaatccagctcctcccagcagaacaacacagtcctggtggaaggctgcttctgtcctgagggcaccatgaactacgctcctggctttgatgtctgcgtgaagacctgcggctgtgtgggacctgacaatgtgcccagagagtttggggagcacttcgagttcgactgcaagaactgtgtctgcctggagggtggaagtggcatcatctgccaacccaagaggtgcagccagaagcccgttacccactgcgtggaagacggcacctacctcgccacggaggtcaaccctgccgacacctgctgcaacattaccgtctgcaagtgcaacaccagcctgtgcaaagagaagccctccgtgtgcccgctgggattcgaagtgaagagcaagatggtgcctggaaggtgctgtcccttctactggtgtgagtccaagggggtgtgtgttcacgggaatgctgagtaccagcccggttctccagtttattcctccaagtgccaggactgcgtgtgcacggacaaggtggacaacaacaccctgctcaacgtcatcgcctgcacccacgtgccctgcaacacctcctgcagccctggcttcgaactcatggaggcccccggggagtgctgtaagaagtgtgaacagacgcactgtatcatcaaacggcccgacaaccagcacgtcatcctgaagcccggggacttcaagagcgacccgaagaacaactgcacattcttcagctgcgtgaagatccacaaccagctcatctcgtccgtctccaacatcacctgccccaactttgatgccagcatttgcatcccgggctccatcacattcatgcccaatggatgctgcaagacctgcacccctcgcaatgagaccagggtgccctgctccaccgtccccgtcaccacggaggtttcgtacgccggctgcaccaagaccgtcctcatgaatcattgctccgggtcctgcgggacatttgtcatgtactcggccaaggcccaggccctggaccacagctgctcctgctgcaaagaggagaaaaccagccagcgtgaggtggtcctgagctgccccaatggcggctcgctgacacacacctacacccacatcgagagctgccagtgccaggacaccgtctgcgggctccccaccggcacctcccgccgggcccggcgctcccctaggcatctggggagcgggtgagcggggtgggcacagcccccttcactgccctcgacagctttacctcccccggaccctctgagcctcctaagctcggcttcctctcttcagatatttattgtctgagtctttgttcagtccttgctttccaataataaactcagggggacatgc

SEQ ID NO: 4 - DNA, 1536 bp, Homo sapiens
  misc_feature (1)..(84): >gi|20149499|ref|NM_000190.2| Homo sapiens hydroxymethylbilane synthase (HMBS), mRNA
gtgacgcgaggctctgcggagaccaggagtcagactgtaggacgacctcgggtcccacgtgtccccggtactcgccggccggagcccccggcttcccggggccgggggaccttagcggcacccacacacagcctactttccaagcggagccatgtctggtaacggcaatgcggctgcaacggcggaagaaaacagcccaaagatgagagtgattcgcgtgggtacccgcaagagccagcttgctcgcatacagacggacagtgtggtggcaacattgaaagcctcgtaccctggcctgcagtttgaaatcattgctatgtccaccacaggggacaagattcttgatactgcactctctaagattggagagaaaagcctgtttaccaaggagcttgaacatgccctggagaagaatgaagtggacctggttgttcactccttgaaggacctgcccactgtgcttcctcctggcttcaccatcggagccatctgcaagcgggaaaaccctcatgatgctgttgtctttcacccaaaatttgttgggaagaccctagaaaccctgccagagaagagtgtggtgggaaccagctccctgcgaagagcagcccagctgcagagaaagttcccgcatctggagttcaggagtattcggggaaacctcaacacccggcttcggaagctggacgagcagcaggagttcagtgccatcatcctggcaacagctggcctgcagcgcatgggctggcacaaccgggtggggcagatcctgcaccctgaggaatgcatgtatgctgtgggccagggggccttgggcgtggaagtgcgagccaaggaccaggacatcttggatctggtgggtgtgctgcacgatcccgagactctgcttcgctgcatcgctgaaagggccttcctgaggcacctggaaggaggctgcagtgtgccagtagccgtgcatacagctatgaaggatgggcaactgtacctgactggaggagtctggagtctagacggctcagatagcatacaagagaccatgcaggctaccatccatgtccctgcccagcatgaagatggccctgaggatgacccacagttggtaggcatcactgctcgtaacattccacgagggccccagttggctgcccagaacttgggcatcagcctggccaacttgttgctgagcaaaggagccaaaaacatcctggatgttgcacggcagcttaacgatgcccattaactggtttgtggggcacagatgcctgggttgctgctgtccagtgcctacatcccgggcctcagtgccccattctcactgctatctggggagtgattaccccgggagactgaactgcagggttcaagccttccagggatttgcctcaccttggggccttgatgactgccttgcctcctcagtatgtgggggcttcatctctttagagaagtccaagcaacagcctttgaatgtaaccaatcctactaataaaccagttctgaaggtaaaaaaaaaaaaaaaaaaaaaaaaaaaaaaaaaaa

SEQ ID NO: 5 - DNA, 20 bp, Artificial
  Artificial
atgaactacgctcctggctt

SEQ ID NO: 6 - DNA, 21 bp, Artificial
  Artifical
aaactctctgggcacattgtc

SEQ ID NO: 7 - DNA, 21 bp, Artificial
  Artifical
cgtgaagacctgcggctgtgt

SEQ ID NO: 8 - DNA, 21 bp, Artificial
  Upper Primer
accacagtcacgacgatcaca

SEQ ID NO: 9 - DNA, 19 bp, Artificial
  Lower primer
cggggttggagttgttgct

SEQ ID NO: 10 - DNA, 24 bp, Artificial
  Probe
ctatgcagagccacccaaaccctt

SEQ ID NO: 11 - DNA, 22 bp, Artificial
  Artificial
cacccttcagggtcttgagatt

SEQ ID NO: 12 - DNA, 20 bp, Artificial
  Artificial
tccgtttctgccagtgtgtc

SEQ ID NO: 13 - DNA, 24 bp, Artificial Sequence
  modified_base (1)..(20): Probe 5′ Quasar 570 d
acagctgagcatgaaagctgcctt

SEQ ID NO: 14 - DNA, 22 bp, Artificial Sequence
  modified_base (1)..(9): Primer 1
ctgcttcgctgcatcgctgaaa

SEQ ID NO: 15 - DNA, 22 bp, Artificial
  Artificial
cagactcctccagtcaggtaca

SEQ ID NO: 16 - DNA, 30 bp, Artificial
  Artificial
cctgaggcacctggaaggaggctgcagtgt

(19) United States
(12) Patent Application Publication
(10) Pub. No.: US 2007/0281305 A1
(43) Pub. Date: Dec. 6, 2007

(54) Detection of lymph node metastasis from gastric carcinoma

(76) Inventors: Sean Wuxiong Cao, Three Bridges, NJ (US); George Green, Newton, NJ (US); Jennifer Jones, Morris Plains, NJ (US); Ming Xu, Berkeley Heights, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/447,255

(22) Filed: Jun. 5, 2006

(51) Int. Cl. C12Q 1/68; G01N 33/574

(52) U.S. Cl. 435/6; 435/7.23

(57) ABSTRACT

Methods, compositions and articles are directed to diagnosing the lymph node metastasis from gastric carcinoma, differentiating between negative lymph nodes and lymph nodes with metastasis from gastric carcinoma, testing tissue samples of for determining gastric carcinoma staging status.

### BACKGROUND OF THE INVENTION

There are about 876,000 new stomach cancer cases each year worldwide (Shibuya, Mathers et al. 2002). In the United States, the estimated new cases and deaths in 2005 for stomach cancer are 21,860 and 11,550 respectively (Jemal, Murray et al. 2005). Gastric carcinoma is the most common type (90% to 95%) of stomach cancer, which is followed in the order of incidence by lymphomas, carcinoids and mesenchymal spindle cell tumors (Kumar, Cotran et al. 2003).

Complete local regional control is the key treatment for surviving gastric cancer, which includes total or subtotal gastrectomy, sentinel lymph node dissection, extended lymph node dissection and resection of adjacent tissues affected by metastasis such as splenectomy and pancreatectomy (Hartgrink and van de Velde 2005; Kitagawa, Fujii et al. 2005). Out of all the patients who suffer relapse after curative surgery, up to 87.5% of them are caused by local recurrence or remained regional lymph node metastasis (Songun, Bonenkamp et al. 1996).

In a study covering 148 patients with gastro-esophageal junction (GEJ) carcinomas, it was concluded that the presence of micro metastasis in local lymph nodes is a key indicator of a poor prognosis (Heeren, Kelder et al. 2005). The current prevalent staging/diagnosis of lymph node metastasis from gastric cancer is achieved by palpation and intra-operative frozen section examination. Frozen section examination of lymph nodes can exhibit sensitivity of 74% (Kitagawa, Fujii et al. 2005).

With the appropriate gene markers, gene expression analysis and detection technology such as RT-PCR or quantitative PCR could provide an alternative method for the diagnosis or prognosis of gastric cancer. These methods have potential to provide better sensitivity and accuracy for metastasis detection test. The previously reported gene markers include CK19 (Matsuda, Kitagawa et al. 2004), Carcinoembryonic antigen (CEA), cytokeratin-20 (CK-20), and MAGE-3 (Okada, Fujiwara et al. 2001), CK18, MUCI, hTRT (Ajisaka and Miwa 2003). Unfortunately, as published data shows, most of them are not consistently expressed in all of the tumor tissues and tumor cell lines tested. Markers could not be detected by RT-PCR in 15% (CEA) to over 70% (MAGE-3) of the lymph nodes with metastasis from gastric cancer confirmed by histological examination (Okada, Fujiwara et al. 2001). CK20 was used as a marker for RT-PCR tests for disseminated cancer cells in blood from pancreas, colon, stomach, and lung carcinoma (Chausovsky, Luchansky et al. 1999; Chen, Chen et al. 2004).

Here, a combination of Markers are identified that are consistently expressed in lymph node with metastasis from gastric carcinoma at easily detectable levels which can be differentiated from their background expression in normal lymph nodes. This combination of markers is useful for the detection of disseminated carcinoma cells from gastric carcinoma, or carcinoma of similar cell origin, such as carcinoma of the esophagus, pancreas, and intestine.

### SUMMARY OF THE INVENTION

In one aspect of the invention, a set of Markers is used in combination to detect lymph node metastasis from gastric carcinoma. In lymph nodes with metastasis from gastric carcinoma, the expression levels of these Markers are distinctively higher than those of normal lymph nodes. The combination of Markers are CK19, CEA and MUC-2 markers.

In another aspect of the invention, methods of diagnosing lymph node metastasis from gastric carcinoma involve obtaining biological samples from a patient and measuring the expression levels of genes selected from a group of Markers, where the gene expression levels above pre-determined cut-off levels are indicative of metastasis from gastric carcinoma. Preferably, the biological samples are lymph nodes samples.

In another aspect of the invention, Markers are used in combination in assays to detect lymph node metastasis from gastric carcinoma.

In yet another aspect of the invention, Markers are assayed intraoperatively. The intraoperative assays can be conducted by a method that includes the steps of: preparing RNA from a lymph node that drains from the stomach; performing a quantitative RT-PCR method specific to one or more Markers and determining if the presence of the Marker exceeds a predetermined cut-off.

The present invention encompasses microarray or gene chips for performing the methods provided herein as well as kits and articles of manufacture for conducting the assays.

### DETAILED DESCRIPTION

The inventive methods, compositions, articles, and kits described and claimed in this specification include a combination of Markers. “Marker” is used throughout this specification to refer to:
a) Genes and gene expression products such as mRNA and corresponding cDNA, peptides, proteins, fragments and complements of each of the foregoing, and
b) Compositions such as probes, antibodies, ligands, haptens, and labels that, through physical or chemical interaction with a) indicate the expression of the gene or presence of the gene expression product and wherein the gene, gene expression product or compositions correspond with SEQ ID NO NM_{—}002276, SEQ ID NO NM_{—}002457, and SEQ ID NO M17303.

A gene corresponds to the sequence designated by a SEQ ID NO when it contains that sequence. A gene segment or fragment is also said correspond to the sequence of such gene when it contains a portion of the referenced sequence or its complement sufficient to distinguish it as being the sequence of the gene. A gene expression product is additionally said to correspond to such sequence when its RNA, mRNA, or cDNA hybridizes to the composition having such sequence (e.g. a probe) or, in the case of a peptide or protein; it is encoded by such mRNA. A segment or fragment of a gene expression product corresponds to the sequence of such gene or gene expression product when it contains a portion of the referenced gene expression product or its complement sufficient to distinguish it as being the sequence of the gene or gene expression product.

The Markers of this invention correspond to genes that encode three well-known proteins: carcinoembryonic antigen (SEQ ID NO M17303), mucin 2 (SEQ ID NO NM_{—}002457), and cytokeratin 19 (SEQ ID NO NM_{—}002276). Individually, the genes when used as cancer markers have not enabled an assay with acceptable performance, particularly with respect to intraoperative uses. However, in combination, as is shown in the Examples, the Markers provide sensitive and specific tests to determine gastric carcinoma metastasis. Controls are generally also assayed to ensure that a negative result does not arise from a faulty run of the assay. Constitutively expressed genes such as PBGD (SEQ ID NO NM_{—}000190) are useful in this regard and reagents for assaying them are preferably included in kit components. PBGD is preferred because, among other things, it contains no known pseudogenes in humans, it is constitutively expressed in human tissues and it is expressed at a relatively low level and therefore is less likely to cause inhibition of the amplification of target sequences of interest.

Sample preparation generally involves working with excised tissue, preferably lymph node tissue. The distribution of metastases and micrometastases in tissues is not uniform in nodes or other tissues. Therefore, a sufficiently large sample should be obtained so that metastases will not be missed. One approach to the sampling issue in the present method is to homogenize a large tissue sample, and subsequently an appropriate portion of the homogenate is to be used to prepare RNA for molecular testing.

Preferably, the samples used in the methods of the invention are lymph node samples from lymph nodes that drain from the stomach. A useful procedure for preparing such a sample for RT-PCR assay follows; an Omni homogenizer and disposable probe for homogenization are used followed by purification of RNA with RNeasy (Qiagen) kit reagents:
a) Determine the sample weight in milligrams, if not previously recorded. Place a fresh piece of wax paper on a balance, tare, and weigh the sample. Preferably, nodes samples are greater than 30 mg.
b) Using a fresh scalpel, mince all tissue from the same node into pieces approximately 4 mm in diameter. Care is taken to avoid contamination of the tissue during processing.
c) Add homogenization (RLT) buffer to the polypropylene homogenization tube. Preferably, use 2 mL homogenization buffer per 100 mg tissue.
d) Using a clean forceps, transfer the tissue into the homogenization buffer.
e) Place a new homogenization probe onto the manual homogenizer.
f) Homogenize each node completely.
g) Process the homogenate per the RNA Purification procedures in standard Qiagen protocol, RNA is stored at −65° C. or below before use.
h) Dispose of the homogenization probe.
i) Store any remaining homogenate at −65° C. or below for possible later use.

Diagnostic assays can then be conducted. The Markers of this invention are useful for providing important clinical information such as whether the gastric carcinoma has metastasized to lymph node, which is indicative to the clinical staging of gastric carcinoma and the prognosis for the patient.

Collectively, this information is referred to as “Diagnostic Information” in this specification. Additionally, such Diagnostic Information includes its use as a component in providing therapy. For example, a physician having such Diagnostic Information can base his therapy (e.g., adjuvant therapy) on an indication that the cancer has metastasized to lymph nodes.

Preferred methods for assaying the Markers of the invention include determining the amount of mRNA that is produced by a gene that can code for a protein or peptide. This can be accomplished by competitive reverse transcriptase PCR (RT-PCR), real time RT-PCR, Northern Blot analysis or other tests with similar capabilities. Amplified complementary RNA (cRNA) can also be prepared from mRNA (RNA) and then applied to DNA microarrays and other evolving assay formats for testing and analysis. A number of different array configurations and methods for their production are known to those of skill in the art and are described in U.S. patents such as: U.S. Pat. Nos. 5,445,934; 5,532,128; 5,556,752; 5,242,974; 5,384,261; 5,405,783; 5,412,087; 5,424,186; 5,429,807; 5,436,327; 5,472,672; 5,527,681; 5,529,756; 5,545,531; 5,554,501; 5,561,071; 5,571,639; 5,593,839; 5,599,695; 5,624,711; 5,658,734; and 5,700,637. The most well accepted measurements used to quantitate the results of a PCR is the Ct (threshold cycle) determination where Ct is defined as as the cycle number at which the fluorescence emission exceeds a fixed threshold. This are readily determinable according to well known procedures with algorithms for determining Ct set in the software of most PCR analyzers. An example of this is embodied in the SmartCycler PCR Instrument from Cepheid Corporation which is a preferred analytical platform.

Microarray technology allows for the measurement of the steady-state mRNA level of genes. The product of these analyses are typically measurements of the intensity of the signal received from a probe set used to detect a target sequence prepared from the sample that hybridizes to a nucleic acid sequence at a known location on the microarray. Typically, the intensity of the signal is proportional to the quantity of target prepared, and thus mRNA, expressed in the sample cells. A large number of such techniques are available and useful. Preferred methods for determining gene expression can be found in U.S. Pat. Nos. 6,271,002; 6,218,122; 6,218,114; and 6,004,755.

Analysis of the expression levels of genes can be conducted by comparing signal intensities of microarrays. This is best done by generating a ratio matrix of the expression intensities of genes in a test sample versus those in a control sample. For instance, the gene expression intensities from a diseased tissue can be compared with the expression intensities generated from benign or normal tissue of the same type. A ratio of these expression intensities indicates the fold-change in gene expression between the test and control samples. The invention also encompasses the above methods where the comparison of expression patterns is conducted with pattern recognition methods. Levels of up and down regulation can be distinguished based on fold changes of the intensity measurements of hybridized microarray probes. A 1.5 fold difference is preferred for making such distinctions (or a p-value less than 0.05). That is, before a gene is said to be differentially expressed in diseased versus normal cells, the diseased cell is found to yield at least about 1.5 times more, or 1.5 times less intensity than the normal cells. The greater the fold difference, the more preferred is use of the gene as a diagnostic or prognostic tool. Genes selected for the combination of this invention have expression levels that result in the generation of a signal that is distinguishable from those of the normal or non-modulated genes by an amount that exceeds background using clinical laboratory instrumentation. The present invention encompasses microarrays or gene chips for performing the methods provided herein. The microarrays can contain isolated nucleic acid sequences, their complements, or portions thereof corresponding to Markers where the combination is sufficient to characterize gastric cancer or risk of relapse in a biological sample. The microarray preferably measures or characterizes at least about 1.5-fold over- or under-expression, provides a statistically significant p-value over- or under-expression, or a p-value is less than 0.05. Preferably, the microarray contains a cDNA array or an oligonucleotide array and may contain one or more internal control reagents.

The gene Markers are differentially expressed as up regulated in lymph nodes with metastasis from gastric carcinoma relative to those lymph nodes without metastasis conditions. Up regulation is a relative term meaning that a detectable difference (beyond the contribution of noise in the system used to measure it) is found in the amount of expression of the genes relative to some baseline. In this case, the baseline is the measured gene expression of makers in normal lymph nodes without metastasis from gastric carcinoma. The genes of interest in the tested lymph node samples are then either up regulated or unchanged relative to the baseline level using the same measurement method. A diagnosis or prognosis includes the determination of disease/status issues such as determining the likelihood of relapse and type of therapy. Determining the likelihood of relapse involves comparing the patterns with patterns associated with known relapse and/or known non-relapsing events or exceeding a cutoff value (usually a Ct in the case of PCR) set for such a distinction.

In some embodiments of the invention, the Marker combination of this invention can be used in conjunction with other gene-based Markers and non-genetic diagnostic methods useful in cancer diagnosis, prognosis, or treatment monitoring. For example, in some circumstances it is beneficial to combine the diagnostic power of the gene expression based methods described above with data from conventional markers such as serum protein markers (e.g., Cancer Antigen 27.29 (“CA 27.29”)). This approach can be particularly useful when other testing produces ambiguous results.

The present invention encompasses methods of generating a gastric cancer diagnostic patient report and reports obtained thereby, by obtaining a biological sample from the patient; measuring gene expression of the sample; and using the results obtained thereby to generate the report. The report can also contain an assessment of patient outcome and/or probability of risk relative to the patient population.

The present invention encompasses compositions containing a microarray probe set or PCR primer/probe set corresponding to the Markers. Articles of manufacture according to the invention also include media or formatted assays used to reveal gene expression information. These can comprise, for example, microarrays in which sequence complements or probes are affixed to a matrix to which the sequences indicative of the genes of interest combine creating a readable determinant of their presence. Alternatively, articles according to the invention can be fashioned into reagent kits for conducting hybridization, amplification, and signal generation indicative of the level of expression of the genes of interest for detecting cancer.

Kits made according to the invention include formatted assays. These can include all or some of the materials needed to conduct the assays such as reagents and instructions and a medium through which nucleic acid sequences, their complements, or portions thereof are assayed. Where the assay is to be conducted in another format, the kit will have components peculiar to that format. For example, if the format is an amplification format such as PCR, Rolling Circle Amplification methods (RCA), Ligase Chain Reaction methods (LCR), Strand Displacement Amplification methods (SDA), Nucleic Acid Sequence Based Amplification methods (NASBA), then the kit will contain reagents and materials appropriate to such technologies. Optionally, the kit includes sample preparation reagents and or articles (e.g., tubes) to extract nucleic acids from tissue such as lymph node tissue. The kits may also include articles to minimize the risk of sample contamination (e.g., disposable scalpel and surface for lymph node dissection and preparation).

In a preferred kit, reagents necessary for a one-tube QRT-PCR process are included such as reverse transcriptase, a reverse transcriptase primer, a corresponding PCR primer set (for Markers and, preferably, controls), a thermostable DNA polymerase, such as Taq polymerase, and a suitable detection reagent(s), such as, without limitation, a scorpion probe, a probe for a fluorescent 5′nuclease assay, a molecular beacon probe, a single dye primer or a fluorescent dye specific to double-stranded DNA, such as ethidium bromide. The primers are preferably in quantities that yield high concentrations. Thermostable DNA polymerases are commonly and commercially available from a variety of manufacturers. Additional materials in the kit may include: suitable reaction tubes or vials, a barrier composition such as a wax bead, optionally including magnesium; reaction mixtures (typically 10×) for the reverse transcriptase and the PCR stages, including necessary buffers and reagents such as dNTPs; nuclease-or RNase-free water; RNase inhibitor; control nucleic acid (s) and/or any additional buffers, compounds, co-factors, ionic constituents, proteins and enzymes, polymers, and the like that may be used in reverse transcriptase and/or PCR stages of QRT-PCR reactions. Optionally, the kits include nucleic acid extraction reagents and materials.

The following examples are provided to illustrate but not limit the claimed invention. All references cited herein are hereby incorporated by reference herein.

### EXAMPLE 1

This example is based on the use of real-time PCR. In real-time PCR, the products of the polymerase chain reaction are monitored in real-time during the exponential phase of PCR rather than by an end-point measurement. Hence, the quantification of DNA and RNA is much more precise and reproducible. Fluorescence values are recorded during every cycle and represent the amount of product amplified to that point in the amplification reaction. The more templates present at the beginning of the reaction, the fewer number of cycles it takes to reach a point in which the fluorescent signal is first recorded as statistically significant above background, which is the definition of the (Ct) values. The concept of the threshold cycle (Ct) allows for accurate and reproducible quantification using fluorescence based RT-PCR. Homogeneous detection of PCR products are preferably performed based on: (a) double-stranded DNA binding dyes (e.g., SYBR Green), (b) fluorogenic probes (e.g., Taq Man probes, Molecular Beacons), and (c) direct labeled primers (e.g., Amplifluor primers).

A total of 24 H&E metastatic positive lymph nodes and 18 H&E metastatic negative lymph node were obtained from patients diagnosed with gastric carinoma. The lymph nodes were selected as those that would drain from the stomach. 30 mg pieces of tissue were cut from each node.

A 30 mg piece of each lymph node tissue was processed by as follows:
1. The tissue piece was added to 2 ml of Buffer RLT and homogenized manually for 20-40 sec by means of a disposable tissue grinder.
2. Mix 400 μL of homogenate with 400 μL of 70% ethanol in a 4.5 mL tube by vortexing for 10 seconds.
3. For each sample, attach a VacValve onto a Vacuum Manifold, and a disposable VacConnector to each valve.
4. Attach a RNeasy mini column on to the VacConnector, leaving the cap open.
5. Aliquot 700 μL homogenate/ethanol mix from step 2 onto the RNA Spin Column.
6. Turn VacValves to the On position and apply vacuum (800-1200 mbars) until sample is filtered (approximately 30 seconds).
7. Stop vacuum; add 700 μL of Wash Buffer 1 to the Column. Start vacuum and allow the solution to filter through the Column. Stop vacuum.
8. Add 700 μL of Wash Buffer 2 to the Column. Start vacuum and allow the solution to filter through the column. Stop vacuum.
9. Remove each Column from the Vacuum Manifold and place into a 2 mL collection tube (supplied with column).
10. Centrifuge the tubes containing the RNA Spin Columns for 30 seconds at >10,000 RPM in a microcentrifuge.
11. Discard the collection tube. Put the Column into a new fresh collection tube (1.5-1.7 mL polypropylene microcentrifuge tube).
12. Add 50 μL of RNase-free water directly to the filter membrane of Column.
13. Centrifuge at >10,000 RPM for 30 seconds in a microcentrifuge.
14. Before continuing to step 14, ensure that the collection tube has been labeled in such a way that the source specimen can be identified (example: patient ID& Node ID).
15. Discard the Column. Approximately 50 μL of eluted RNA solution will be contained in the collection tube.

Extracted RNA was subjected to one-step RT-PCR amplification in a Cepheid Smart Cycler II. The reaction contains the following components: 50 mM Bicine/KOH, pH 8.2, 115 mM Potassium Acetate, 8% v/v Glycerol, 0.2 mg/ml BSA, 150 mM Trehalose, 0.2% v/v Tween 20, 0.2 mM Tris-Cl pH 8, 3.5 mM MnSO4 or Mn (acetate), 0.3 mM dATP, 0.3 mM dCTP, 0.3 mM dGTP, 0.3 mM TTP, 100 U/ml Tth, 20 ug/ml Antibody TP6-25.3, 0.4-0.8 uM each probe and primer. Probe and primer sequences are designed such that non-specific priming events are minimized and such that they are compatible with multiplexed amplification/detection with the following rapid RT-PCR profile: 95 C for 3 sec, 60 C for 3 min, then 32 cycles of: 95 C for 1 sec, 65 C for 6 sec* and 72 C for 3 sec−total amplification time=16 min.

The primers and probes used in this example were as follows: MUC2:

(SEQ ID NO: 5: NM_002457)

Upper primer:
ATGAACTACGCTCCTGGCTT

(SEQ ID NO: 6: NM_002457)

Lower primer:
AAACTCTCTGGGCACATTGTC

(SEQ ID NO: 7: NM_002457)

Probe:
FAM-CGTGAAGACCTGCGGCTGTGT-BHQ1-TT
CEA:

(SEQ ID NO: 8: M17303)

Upper primer:
ACCACAGTCACGACGATCACA

(SEQ ID NO: 9: M17303)

Lower primer:
CGGGGTTGGAGTTGTTGCT

(SEQ ID NO: 10: M17303)

Probe:
TexasRed-CTATGCAGAGCCACCCAAACCCTT-BHQ2-TT
CK19:

(SEQ ID NO: 11)

Upper primer:
CACCCTTCAGGGTCTTGAGATT

(SEQ ID NO: 12)

Lower primer:
TCCGTTTCTGCCAGTGTGTC

(SEQ ID NO: 13)

Probe:
5′ Quasar 570
d(ACAGCTGAGCATGAAAGCTGCCTT)-BHQ-2-TT) 3′
PBGD:

(SEQ ID NO: 14)

Primer 1
CTGCTTCGCTGCATCGCTGAAA

(SEQ ID NO: 15)

Primer 2
CAGACTCCTCCAGTCAGGTACA

(SEQ ID NO: 16)

Probe
FAM-CCTGAGGCACCTGGAAGGAGGCTGCAGTGT-TAMRA

The samples and the assay results were summarized in Table 1. The resulting Ct values were utilized to determine relative expression of the genes across all samples tested. The Ct value established was 27 for the Markers and 35 for the internal control (PBGD). Samples with a Ct of 27 or lower for any of the 3 markers are considered positive for metastasis, while samples with Ct values above 27 for all 3 markers are considered negative for metastasis. The Ct value for internal control needs to be lower than 35 for the negative diagnosis result to be valid. Based on these cut-off values, the assay was used to correctly evaluate 17 out of the 18 negative lymph nodes (94.4% specificity) and all 24 of the positive lymph nodes (100% sensitivity).

TABLE 1
Detection of lymph node metastasis from
patients with gastric adenocarcinoma

| | Lymph Node | | | | |
|---|---|---|---|---|---|
| Sample | Diagnosis | IC Ct | MUC Ct | CK19 Ct | CEA Ct |
| 1 | Normal | 25.65 | 36.6 | 27.25 | 27.9 |
| 2 | Normal | 25.45 | 30.6 | 18.8 | 23.15 |
| 3 | Normal | 23.7 | 32.8 | 31.2 | 33.5 |
| 4 | Normal | 24.8 | 40 | 40 | 40 |
| 5 | Normal | 25.2 | 40 | 33.3 | 40 |
| 6 | Normal | 24.4 | 40 | 31.7 | 40 |
| 7 | Normal | 24.8 | 31.8 | 30.5 | 33.9 |
| 8 | Normal | 25.2 | 40 | 31.2 | 40 |
| 9 | Normal | 26.1 | 35.4 | 40 | 40 |
| 10 | Normal | 27.4 | 34.5 | 34.6 | 40 |
| 11 | Normal | 24.7 | 28.2 | 28 | 28.9 |
| 12 | Normal | 25.7 | 27.3 | 28.5 | 29.1 |
| 13 | Normal | 25.8 | 40 | 32.8 | 32.7 |
| 14 | Normal | 26.2 | 40 | 31 | 40 |
| 15 | Normal | 19.2 | 40 | 37.4 | 40 |
| 16 | Normal | 26.4 | 40 | 35.7 | 40 |
| 17 | Normal | 27.2 | 40 | 32.9 | 40 |
| 18 | Normal | 26.3 | 40 | 32.7 | 40 |
| 19 | Metastatic | 23.6 | 23.9 | 21.05 | 21.35 |
| 20 | Metastatic | 23.85 | 19.05 | 19.65 | 19.5 |
| 21 | Metastatic | 21.15 | 21 | 19.3 | 19.2 |
| 22 | Metastatic | 26.1 | 22.45 | 20.1 | 20.7 |
| 23 | Metastatic | 25.25 | 24.8 | 18.65 | 20.35 |
| 24 | Metastatic | 31.85 | 26.7 | 24.75 | 24.4 |
| 25 | Metastatic | 30.3 | 26.25 | 23.45 | 25.65 |
| 26 | Metastatic | 26.7 | 22.1 | 20.95 | 23.9 |
| 27 | Metastatic | 27.7 | 24.55 | 23.5 | 28.3 |
| 28 | Metastatic | 26.15 | 32.6 | 25.55 | 30.3 |
| 29 | Metastatic | 25.85 | 24.95 | 19.55 | 24.45 |
| 30 | Metastatic | 25.75 | 24.65 | 19.45 | 23.8 |
| 31 | Metastatic | 29.05 | 22.85 | 18.5 | 21.5 |
| 32 | Metastatic | 27.7 | 24.95 | 20.2 | 24.25 |
| 33 | Metastatic | 27.8 | 27 | 21.5 | 21.05 |
| 34 | Metastatic | 25.15 | 25.25 | 25.8 | 19.95 |
| 35 | Metastatic | 26.6 | 23.05 | 23.5 | 22.1 |
| 36 | Metastatic | 27.65 | 32.55 | 17.05 | 16.05 |
| 37 | Metastatic | 24.95 | 26.35 | 19.25 | 17.35 |
| 38 | Metastatic | 24.75 | 25.4 | 18.2 | 15.2 |
| 39 | Metastatic | 25.8 | 28 | 21.85 | 19.3 |
| 40 | Metastatic | 28.1 | 31.95 | 20.35 | 18.15 |
| 41 | Metastatic | 25.25 | 18.8 | 19.45 | 18.05 |
| 42 | Metastatic | 25 | 32.9 | 19.2 | 19.5 |

1. A method of diagnosing lymph node metastasis from gastric carcinoma comprising the steps:
a) obtaining a biological sample from a patient, and
b) measuring Marker levels in the sample;
wherein the levels above the pre-determined cut-off levels are indicative of lymph node metastasis from gastric carcinoma.

2. The method of claim 1 wherein the Markers are genes, gene fragments, or complements thereof.

3. The method of claim 1 wherein the Markers are proteins, protein fragments, or polypeptides.

4. The method of claim 1 further comprising the use of another diagnostic method.

5. The method of claim 4 wherein the other diagnostic method comprises conducting a serum assay.

6. The method of claim 1 further comprising the measurement of a consitutively expressed gene.

7. The method of claim 1 having a sensitivity of at least 80%.

8. The method of claim 1 having a specificity of at least 70%.

9. The method of claim 1 wherein the sample comprises lymph node tissue.

10. A method of determining or adjusting therapy for a gastric carcinoma patient comprising the steps:
a. obtaining a biological sample from a gastric cancer patient; and
b. measuring the levels in the sample of a Marker;
wherein levels above or below a cut-off levels are consistent with a course of disease or physical condition for which a therapy is determined or adjusted.

11. The method of claim 10 wherein the Marker is a gene, a gene fragment, or the complement thereof.

12. The method of claim 10 wherein the Marker is a protein, protein fragment, or polypeptide.

13. The method of claim 10 further comprising the measurement of a consitutively expressed gene.

14. The method of claim 10 wherein the specificity is at least 70%.

15. The method of claim 10 wherein the sensitivity is at least about 80%.

16. The method of claim 1 conducted with a microarray.

17. The method of claim 10 conducted with a microarray.

18. The method of claim 1 conducted by using an amplification process.

19. The method of claim 18 wherein the amplification process is a PCR.

20. The method of claim 19 wherein the PCR s an RT-PCR.

21. The method of claim 10 conducted by using an amplification process.

22. A kit for conducting an assay to determine gastric cancer diagnosis in a biological sample comprising materials for detecting a Marker.

23. The kit of claim 22 including a microarray and reagents.

24. The kit of claim 22 including PCR reagents.

25. The kit of claim 22 including instructions.

26. The kit of claim 22 wherein the Markers consist of CEA, MUC2, and CK-19.

27. A method of diagnosing lymph node metastasis from gastric carcinoma comprising the steps:
a) obtaining a biological sample from a patient, and
b) measuring Marker levels in the sample;
wherein the levels above the pre-determined cut-off levels are indicative of lymph node metastasis from gastric carcinoma and wherein the Markers consist of CEA, MUC2, and CK-19.

28. The method of claim 27 further comprising the use of another diagnostic method.

29. The method of claim 27 wherein the other diagnostic method comprises conducting a serum assay.

30. The method of claim 27 further comprising the measurement of a consitutively expressed gene.

31. The method of claim 27 having a sensitivity of at least 80%.

32. The method of claim 27 having a specificity of at least 70%.
